(19) United States
(12) Patent Application Publication
(10) Pub. No.: US 2008/0097494 A1
(43) Pub. Date: Apr. 24, 2008

(54) Fluid port for an adjustable gastric banding system

(76) Inventors: Brian B. Quebbemann, Newport Beach, CA (US); Clark B. Foster, Mission Viejo, CA (US)

Correspondence Address: LAW OFFICES OF MORLAND C FISCHER, 2030 MAIN ST, SUITE 1300, IRVINE, CA, 92614, US

(21) Appl. No.: 11/513,395

(22) Filed: Aug. 31, 2006

(51) Int. Cl. A61B 17/12

(52) U.S. Cl. 606/157

(57) ABSTRACT

An improved fluid port for use in an adjustable gastric banding system that can be laparoscopically-implanted around the upper stomach of an obese patient as part of a procedure to help in weight reduction. The improved fluid port is coupled to a conventional constriction collar of the banding system by way of bio-compatible fluid tubing. The fluid port includes a rigid liner having a needle cannula receiving fluid reservoir at one end thereof at which fluid is added to or withdrawn from the constriction collar and a reservoir extension at the opposite end to be mated to the fluid tubing. The fluid reservoir and the reservoir extension are aligned with one another at an angle so as to be oriented following implantation (e.g., below the xiphoid bone where layers of fat and muscle are minimal) to be quickly and easily located by palpating the patient's skin at the bottom of the sternum immediately below the xyphoid process. A stress relief collar surrounds the connection of the reservoir extension of the inner liner to the fluid tubing so as to reduce fatigue and avoid a possible fracture, whereby to enhance the reliability of the fluid port.

### BACKGROUND OF THE INVENTION

1. Field of the Invention

This invention relates to an improved fluid port for use in a gastric banding system that is suitable to be laparoscopically-implanted around the upper stomach of an obese patient during a procedure to help in weight reduction. The improved fluid port has a configuration that establishes an easy-to-locate target area, following implantation, for receipt of a needle cannula to permit fluid to be injected into or removed from a constriction collar of the banding system to selectively adjust the occlusive pressure generated thereby.

2. Background Art

Gastric banding systems are known which have been surgically implanted around the stomach of an obese patient to generate an occlusive pressure by which to create an early sensation of being full and thereby reduce food intake as part of a weight reduction program. A gastric banding system is commonly implanted with a fluid port at which fluid can be supplied to or removed from an inflatable (i.e., adjustable) fluid chamber of a constriction collar that surrounds the upper stomach. When fluid is added to the fluid chamber at the fluid port, the occlusive pressure around the stomach is correspondingly increased and the diameter of the circular opening is reduced. When fluid is removed, the occlusive pressure is reduced and the diameter is increased.

Several problems persist with most known fluid port devices as a consequence of using a conventional fluid port with a gastric banding system that is surgically implanted within the abdominal cavity of an obese patient. For example, stress and fatigue are known to be concentrated along the tapered transition from the port body to the cannula and at the point where the conventional fluid port is coupled to a fluid line that communicates with the inflatable fluid chamber of the constriction collar. Such stress and fatigue have resulted in a fracture, a leak of fluid into the patient's abdominal cavity, and an ultimate failure of the banding system. The patient may then be subjected to a new surgery in order to repair or replace the banding system or its fluid tubing.

Another significant problem is the difficulty faced by clinical personnel when trying to detect and access the conventional fluid port through the fatty tissue of the obese patient so as to be able to properly insert a needle cannula therein. That is to say, the fluid port is typically located below several inches of fatty tissue and outside the muscular layer of the obese patient. Thus, it has proven difficult for a surgeon to find a firm tissue area to which the fluid port can be sutured so as to minimize movement and provide resistance for cannula insertion. Should an emergency condition arise when it is necessary to immediately reduce the occlusive pressure being generated by the constriction collar around the patient's stomach, emergency room medical personnel have experienced difficulty in being able to quickly find the precise location and orientation of the fluid port for receipt of the cannula. Therefore, some emergency room physicians and other clinicians are reluctant to take prompt action because they are not entirely certain where to insert the cannula. In this case, a very long cannula is often used which may result in the patient's abdominal wall being pierced and his internal organs suffering injury. In another case, the fluid tubing associated with the fluid port could be accidentally punctured and damaged.

If the needle cannula is inserted at the wrong location, time will be wasted and relief will be delayed as the patient is exposed to successive needle sticks until the correct location of the fluid port is finally identified. In this same regard, in order to increase the target area for a successful placement and insertion of a needle cannula through the fatty tissue, the size of the fluid port has had to remain large, which excludes the port from laparoscopic implantation and has resulted in a larger incision to reach the abdominal wall causing more tissue trauma and pain, at the time of surgical implantation.

### SUMMARY OF THE INVENTION

In general terms, this invention relates to an improved fluid port for use in an adjustable gastric banding system that can be laparoscopically-implanted around the upper stomach of an obese patient as part of a procedure to help in weight reduction. The improved fluid port is coupled to a conventional constriction collar of the banding system by way of bio-compatible fluid tubing. The constriction collar includes an inflatable fluid chamber which is removably attached around the stomach of the obese patient and thereby increases or decreases the occlusive pressure applied against the stomach. Thus, the diameter of the lumen of the stomach is either expanded or contracted depending upon whether fluid is added to or withdrawn from the fluid chamber at the improved fluid port via the fluid tubing.

The improved fluid port includes an outer body that is molded around a rigid inner liner which is preferably manufactured from stainless steel, or the like. The inner liner includes a relatively wide fluid reservoir within which a needle cannula is inserted so that fluid can be added to or removed from the inflatable fluid chamber of the constriction collar. The inner liner also includes a relatively narrow reservoir extension projecting outwardly from the fluid reservoir to be mated to the fluid tubing of the banding system. A needle stop plate and a self-healing gel plug are located within the fluid reservoir of the inner liner to limit the movement of the needle cannula therethrough and to automatically close any puncture wounds created by the cannula.

The outer body of the fluid port includes a fluid inlet end to surround the fluid reservoir of the inner liner and a fluid outlet end to surround the reservoir extension. The fluid reservoir and the reservoir extension of the inner liner as well as the fluid inlet and outlet ends of the outer body are aligned relative to one another at an angle (e.g., approximately 135 degrees). In this manner, the rigid inner liner will be configured so that clinicians can quickly and easily palpate and locate the fluid reservoir thereof following implantation so as to be able to accurately insert the needle cannula in order to selectively adjust the occlusive pressure generated by the inflatable fluid chamber of the constriction collar.

The outer body of the fluid port includes attachment wings projecting therefrom to receive surgical sutures for attachment to the patient's tissue (e.g., the facia layer within an area of the abdominal wall characterized by reduced fat and muscle and located adjacent the xiphoid bone at the bottom of the sternum) so as to minimize movement of the fluid port following implantation. The outer body also includes a flat horizontal base extending therearound. During implantation of the fluid port, the flat base is positioned to rest upon the outside of the patient's facia tissue layer (i.e., the upper portion of the linea alba) to which the fluid port is sutured, and the attachment wings are positioned against the opposite inside of the facia tissue layer so that the fluid reservoir of the inner liner will be located in an easily palpable position in the subcutaneous fat and oriented so as to be quickly and easily identified and located by means of palpating the patient's skin below the sternum The ease of locating the fluid port and its proximity to the skin allows a reduction in size such that the port will pass through a small laparoscopic incision.

A strain relief collar extends between the fluid tubing and a taper formed at the fluid outlet end of the outer body of the fluid port. The strain relief collar reduces fatigue and stress buildup at the union of the fluid port and the fluid tubing so as to reduce the possibility of fracture and improve the reliability of the gastric banding system following implantation.

### BRIEF DESCRIPTION OF THE DRAWINGS

FIG. 1 shows a weight reducing gastric banding system that is suitable to be implanted within the abdominal cavity of an obese patient and that includes an improved fluid port according to a preferred embodiment of the present invention;

FIG. 2 shows the gastric banding system of FIG. 1 and an exploded view of the improved fluid port thereof;

FIG. 3 shows the improved fluid port in the assembled configuration and mated to fluid tubing of the gastric banding system;

FIG. 4 is an enlarged detail taken from FIG. 3 showing the union of the improved fluid port to the fluid tubing;

FIG. 5 shows the improved fluid port following implantation within the patient's abdominal cavity to establish an easy-to-locate target area at which to insert the needle cannula of a hypodermic syringe; and

FIG. 6 shows the gastric banding system including the improved fluid port implanted with respect to the patient's anatomy.

### DESCRIPTION OF THE PREFERRED EMBODIMENT

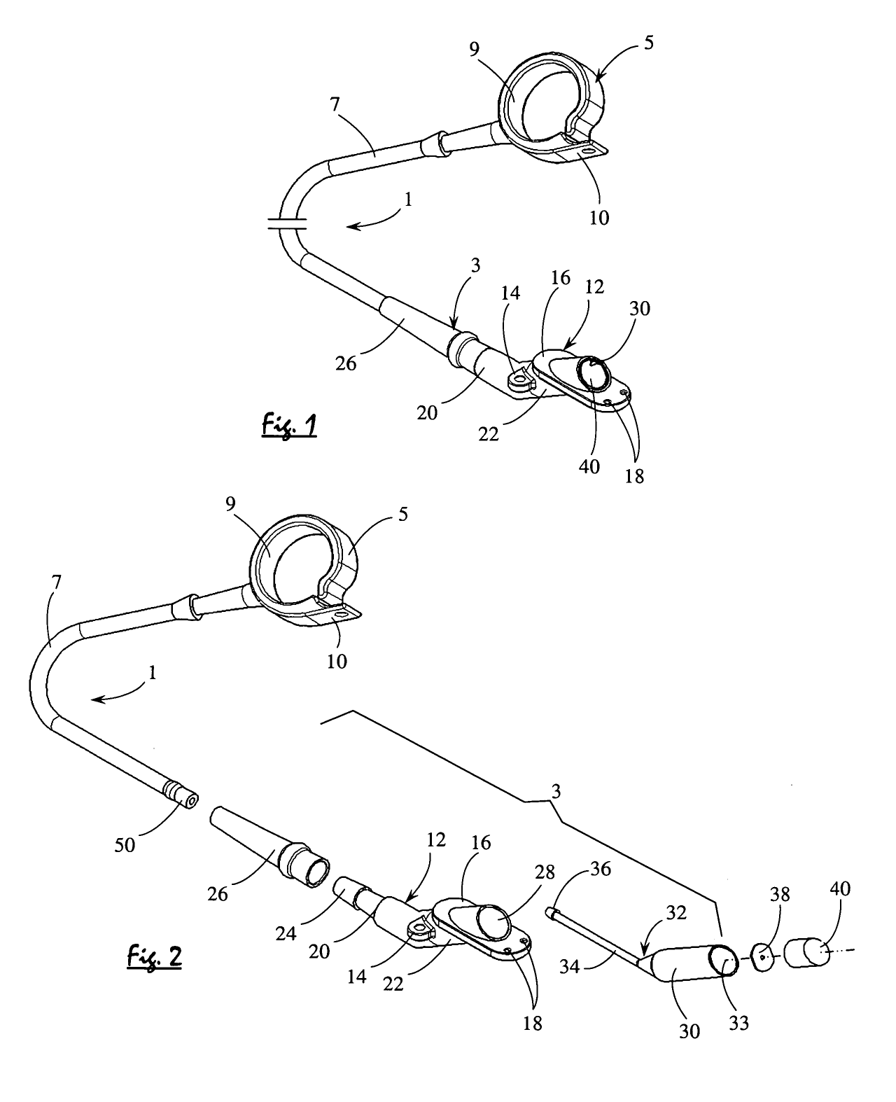

Referring initially to FIGS. 1 and 2 of the drawings, a gastric banding system 1 is shown including the improved fluid port 3 according to a preferred embodiment of this invention. The gastric banding system 1 of FIGS. 1 and 2 has particular application to be laparoscopically-implanted around the upper stomach of an obese patient seeking a procedure to help in weight reduction The fluid port 3 is configured so as to be quickly and easily located following implantation, whereby medical personnel can inject (or remove) a suitable (e.g., saline solution) fluid by which to selectively adjust the occlusive force generated by an inflatable constriction collar 5 of the banding system 1 which is located in surrounding engagement with the stomach of the patient.

Inasmuch as the inflatable constriction collar 5 and its fluid tubing 7 of banding system 1 are commercially available surgical devices, only a brief description thereof will be provided herein. Constriction collar 5, sometimes known as a lap band, includes a hollow, generally circular fluid chamber 9 that is sized to surround the upper portion of an obese patient's stomach. When the fluid chamber 9 is filled with fluid and constricted, a radially-inward occlusive pressure is generated so that food will be trapped in the patient's upper stomach to create an early sensation of being full, whereby to reduce hunger and food intake. A releasable latch 10 enables the fluid chamber 9 of the constriction collar 5 to be closed and removeably secured around the patient's stomach. The fluid tubing 7, which is preferably manufactured from a bio-compatible plastic material, communicates with the fluid chamber 9 to enable the chamber to be infused with fluid when it is necessary to increase constriction and the corresponding occlusive pressure that is applied around the stomach. However, fluid can also be removed from the fluid chamber 9 when it is desirable to reduce the occlusive pressure applied to the patient's stomach, such as in cases of emergency caused by overeating or eating non-prescribed food which results in an upper stomach obstruction.

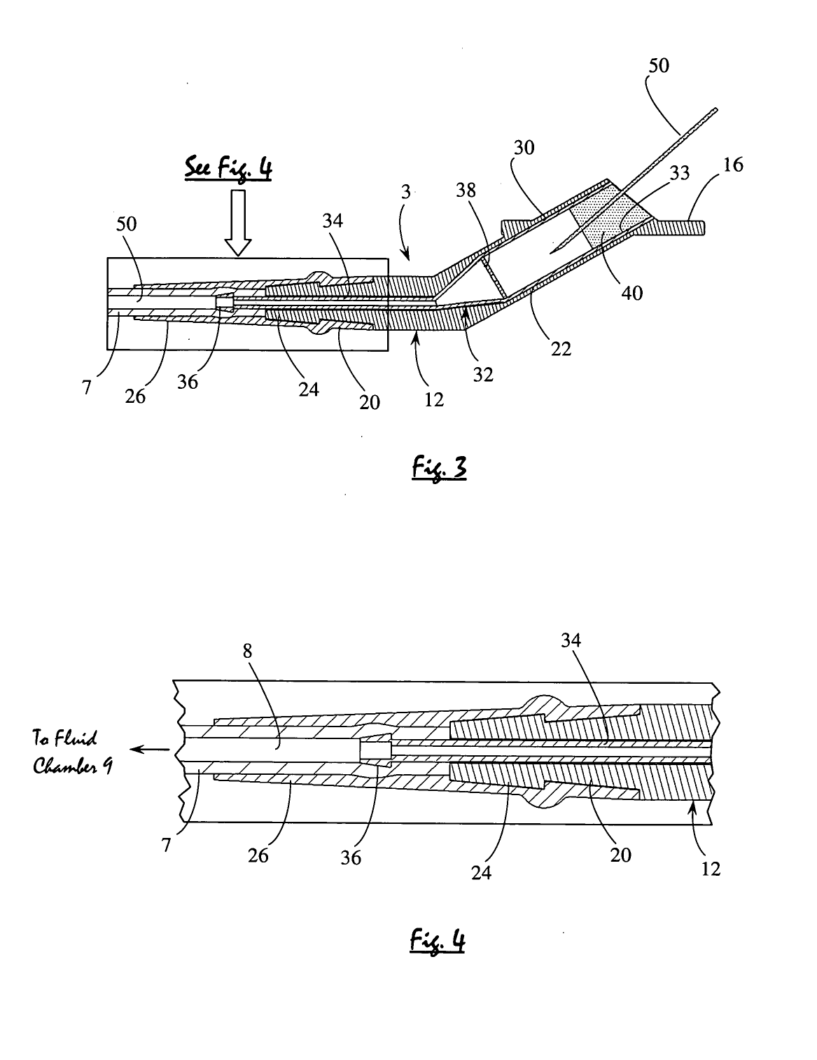

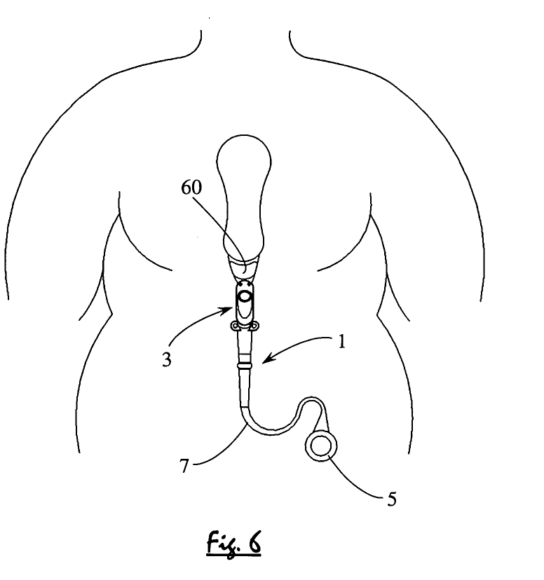

The improved fluid port 3 at which fluid is injected into or removed from the inflatable fluid chamber 9 of constriction collar 5 via tubing 7 includes a one-piece hollow main body 12 that is preferably manufactured from medical grade silicone rubber. Attachment wings 14 (only one of which being visible) project outwardly and in opposite directions from the main body 12 to receive sutures for attachment to a layer of tissue so as to prevent a migration of the fluid port 3 following implantation. The main body 12 includes a flat, horizontally extending base 16 which, as will be disclosed when referring to FIG. 5, is adapted to lay on top of the layer of tissue whereby to automatically orient the fluid port 3 for receipt of a needle cannula (designated 50 in FIG. 3). One or more holes 18 are formed in the base 16 to permit tissue ingrowth or to receive non-dissolving sutures to reinforce the attachment of the fluid port 3 to the patient's tissue and facilitate an easy removal of the fluid port 3 should such be necessary following implantation.

The main body 12 of the fluid port 3 has a fluid outlet end 20 and an opposite fluid inlet end 22. The fluid outlet end 20 and the fluid inlet 22 of the main body 3 are aligned at an angle relative to one another of approximately 135 degrees for an important purpose that will be explained in greater detail hereinafter when referring to FIG. 5. The fluid outlet end 20 has a stepped taper 24 by which to form a close locking fit within a strain relief collar 26. In the assembled configuration of the gastric banding system 1 shown in FIG. 1, one end of the strain relief collar 26 surrounds the stepped taper 24 at the fluid outlet end 20 of main body 12, and the opposite end of strain relief collar 26 surrounds the open (i.e., free) end 8 of fluid tubing 7.

The main body 12 of fluid port 3 is molded around a rigid metal (e.g., stainless steel or any other suitable bio-compatible metal) liner 32. The fluid inlet end 22 of the main body 12 includes a mouth 28 to surround a cylindrical fluid reservoir 30 of the liner 32. The fluid reservoir 30 of liner 32 includes a needle access opening 33 that communicates with a relatively long ad narrow tubular reservoir extension 34. A hose barb 36 is located at the outermost end of the reservoir extension 34.

A needle stop plate 38 (of FIG. 2) is located inwardly of the fluid reservoir 30 of liner 32 to limit the forward movement of the needle cannula (50 of FIG. 3) into the fluid access opening 33 when it is desirable to add fluid to or remove fluid from the inflatable fluid chamber 9 of constriction collar 5 of the banding system 1. In this manner, movement of the cannula 50 can be confined to the reservoir 30 of metal liner 32 so as to avoid damage to the main body 12. Furthermore, the needle stop plate 38 provides a hard surface substantially perpendicular to the needle cannula, such that a clear tactile signal is generated when the needle cannula 50 is inserted into contact with needle stop plate 38. Located within the fluid reservoir 30 of liner 32 ahead of the needle stop plate 38 is a gel plug 40 (of FIG. 1). The gel plug 40 consists of a self-healing hydrophobic gelatinous material that is adapted to automatically seal a puncture wound that is created when the needle cannula 50 is pushed inwardly of the needle access opening 33 of the fluid reservoir 30 of liner 32.

In the assembled configuration of the gastric banding system 1 shown in FIG. 1, the rigid metal line 32 is enclosed within the main body 12 such that the fluid reservoir 30 is surrounded by the fluid inlet end 22 of body 12 and the reservoir extension 34 is surrounded by and projects outwardly from the fluid outlet end 20. In this regard, the fluid reservoir and fluid extension 30 and 34 at opposite ends of liner 32 have the same 135 degree angular alignment with one another as the fluid inlet and outlet ends 22 and 20 of the main body 12.

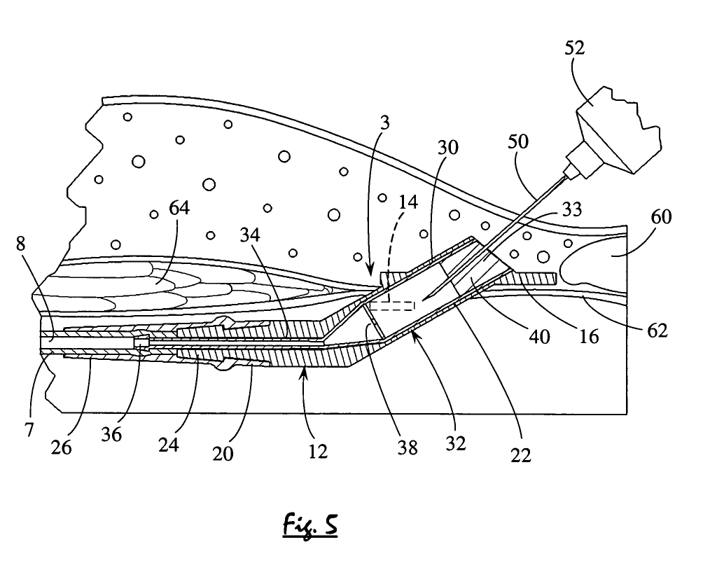

Turning now to FIGS. 3 and 4 of the drawings, in order to connect the improved fluid port 3 of this invention to the fluid tubing 7 so as to be able to communicate with the inflatable fluid chamber 9 of the constriction collar 5 and thereby complete the gastric banding system 1, one end of the strain relief collar 26 is slid over the open end 8 of fluid tubing 7. Next, the hose barb 36 at the outwardly projecting end of the reservoir extension 34 of the rigid metallic liner 32 around which the main body 12 is molded is pushed inwardly of the open end 8 of fluid tubing 7 such that the reservoir extension 34 is captured by and retained within tubing 7. The fluid outlet end 20 of the main body 12 is then pushed inwardly of the opposite end of the strain relief collar 26, such that the stepped taper 24 of fluid outlet end 20 is forced into locked engagement within collar 26.

Accordingly a fluid path is established from the fluid reservoir 30 of the rigid liner 32 to the inflatable fluid chamber 9 of constriction collar 5 (of FIGS. 1 and 2) via the fluid tubing 7 and the reservoir extension 34 of liner 32. Moreover, the needle access opening 33 of fluid reservoir 30 is aligned relative to reservoir extension 34 and oriented to receive the needle cannula 50 of a conventional syringe through the gel plug 40. Hence, fluid can be supplied to or removed from the inflatable fluid chamber 9 at the fluid reservoir 30 depending upon whether the occlusive pressure to be generated by the fluid chamber 9 following implantation of the gastric banding system 1 is to be increased or decreased.

With the fluid port 3 connected to the fluid tubing 7 of the gastric banding system 1, as shown in FIGS. 3 and 4, the strain relief collar 26 surrounds the union of the hose barb 36 at the outermost end of reservoir extension 34 with the open end 8 of fluid tubing 7. By virtue of the foregoing, the strain relief collar 26 reduces the stress where the metal liner 32 meets the biocompatible fluid tubing 7 so as to advantageously avoid inherent fatigue problems that have adversely affected the reliability of conventional gastric banding systems which have employed traditional fluid ports.

FIG. 5 of the drawings shows the improved fluid port 3 implanted within the abdominal cavity of the patient and coupled to the fluid tubing 7 which communicates with the inflatable fluid chamber 9 of the constriction collar 5 (of FIGS. 1 and 2). It is preferable that the fluid port 3 be implanted adjacent the patient's xiphoid bone 60 and below the sternum where minimal muscle and fatty tissue reside between the abdominal wall and the skin. In this case, a physician, surgeon or emergency room worker will be advantageously able to palpate the patient's skin and, by means of using his tactile senses, feel the fluid inlet end 22 of the main body 12 that is molded around the fluid reservoir 30 of the rigid liner 32 so that an infusion target is established for receipt of the needle cannula 50 of a conventional hypodermic syringe 52.

That is, and by virtue of the angled alignment between the fluid reservoir 30 and reservoir extension 34 of the rigid liner 32, the needle access opening 33 of fluid port 3 will be oriented for easy identification to receive the cannula 50 through a relatively thin tissue layer that is characterized by minimal muscle and fat components of an otherwise obese patient. More particularly, the attachment wings 14 of the main body 12 to which surgical sutures are applied to prevent migration of fluid port 3 are seated upon the bottom or inside of the patient's facia tissue layer 62 and the flat, horizontally extending base 16 of the main body 12 is seated upon the opposite top or outside of the facia tissue layer 62.

Accordingly, so as to eliminate bulging at the abdominal wall, while still allowing for ease of palpation and localization and cannula access, only the fluid inlet end 22 of the main body 12 and the targeted needle access opening 33 of the fluid reservoir 30 of rigid liner 32 extend above the facia issue layer 62 and muscle layer 64. The remainder of the fluid port 3, including the reservoir extension 34 of liner 32 and the fluid outlet end 20 of the main body 12 as well as the strain relief collar 26 which surrounds the union of the fluid port 3 with the fluid tubing 7, are disposed below the facia tissue layer 62 and muscle layer 64. In this same regard, with the flat horizontally extending base 16 of the main body 12 lying flush against the outside of facia layer 62, the fluid port 3 will be unable to rotate and protrude downwardly into the abdominal cavity so that damage to the patient's internal organs can be avoided. Likewise, the main body 12 can lay against the inner surface of the abdominal wall so as to avoid intrusion and subsequent damage to the bowel or other viscera.

Turning now to FIG. 6 of the drawings, the improved fluid port 3 of this invention is shown (e.g., laparoscopically) implanted in relation to an obese patient's anatomy and coupled to the conventional constriction collar 5 of gastric banding system 1 via fluid tubing 7. The surgical procedure to place and secure the currently used, conventional fluid port is substantial for both implantation and removal (if required). The typical large incision following surgery is more aesthetically problematic for patients than is the standard laparoscopic incision. To overcome the foregoing, and as was described when referring to FIG. 5, the fluid port 3 is preferably implanted adjacent the xiphoid bone 60 at the bottom of the sternum where layers of fat and muscle are minimal. The incision for implantation is created laparoscopically, using standard laparoscopic techniques, through the peritoneum, pre-peritoneal fat, fascia (linea alba) and into the subcutaneous fat of the upper abdominal midline. In this way, no external incisions need to be made over the site of port implantation. To facilitate fixation of the fluid port 3 to the abdominal wall (at the undersurface of the linea alba), only small puncture wound(s) need to be made for transfascial sutures. Alternatively, the fluid port 3 can be completely fixed in position using standard laparoscopic suturing techniques. Moreover, the patient's rib cage can be used as a guide to enable clinicians to quickly and easily locate (by palpating the patient's skin below the xiphoid bone 60) the target area established by the needle access opening (designated 33 in FIG. 5) at the upstading end of fluid port 3.

What is still more, clinicians will no longer have to make successive needle sticks or use an extra long needle cannula or waste valuable time while attempting to locate the needle target area, especially during emergency situations, as has often occurred as a consequence of implanting a gastric banding system which incorporates a relatively hard-to-locate conventional fluid port. Because the needle target area of the improved fluid port 3 can be more easily and accurately identified, the size of the target area may be advantageously reduced relative to target areas associated with conventional fluid ports. Hence, the improved fluid port 3 can be implanted laparoscopically, thereby eliminating the larger incision and related tissue trauma.

1. For connection to a patient implantable constriction collar to be positioned around a human organ and having an inflatable fluid chamber to which a fluid is added or from which a fluid is removed so as to generate a correspondingly increased or reduced occlusive pressure, and fluid tubing connected to the fluid chamber, the improvement of an implantable fluid port to be coupled to the fluid tubing of the constriction collar at which the fluid is supplied to or withdrawn from the fluid chamber thereof, said fluid port comprising:
an inner liner including a fluid reservoir to receive a needle cannula and a reservoir extension projecting from said fluid reservoir so as to communicate with the fluid tubing of the constriction collar; and
an outer body surrounding said inner line and including a suture attachment area at which to receive a surgical suture for attaching said fluid port to the tissue of the patient and thereby prevent a migration of said fluid port in relation to the constriction collar following implantation.

2. The fluid port recited in claim 1, wherein said inner liner is manufactured from metal and said outer body is manufactured from rubber, said outer body being molded around said inner liner.

3. The fluid port recited in claim 1, wherein the fluid reservoir of said inner liner is cylindrical and said reservoir extension communicating therewith is tubular and sized to be received inwardly of the fluid tubing of the constriction collar, said cylindrical fluid reservoir having a larger diameter than the diameter of said tubular reservoir extension.

4. The fluid port recited in claim 1, wherein the fluid reservoir of said inner liner and said reservoir extension projecting therefrom are aligned at an angle relative to one another.

5. The fluid port recited in claim 4, wherein the fluid reservoir of said inner liner is aligned with said reservoir extension thereof at an angle of approximately 135 degrees.

6. The fluid port recited in claim 1, further comprising a needle stop located within the fluid reservoir of said inner liner to limit the movement of the needle cannula through said fluid reservoir.

7. The fluid port recited in claim 1, further comprising a self-healing gel plug located within the fluid reservoir of said inner liner to automatically close a puncture wound formed when the needle cannula is received through said fluid reservoir.

8. The fluid port recited in claim 1, wherein said outer body also includes an attachment wing by which to form said suture attachment area for receiving a surgical suture for attaching said fluid port to the tissue of the patient following implantation.

9. The fluid port recited in claim 8, wherein said outer body also includes a flat base spaced from said attachment wing, such that said flat base is positioned relative to said attachment wing to lie against one side of the patient's tissue to which said fluid port is attached following implantation, and said attachment wing is positioned to lie at the opposite side of the patient's tissue.

10. The fluid port recited in claim 9, wherein said flat base includes at least one hole through which to receive ingrowth of the patient's tissue for preventing a displacement of said fluid port following implantation.

11. The fluid port recited in claim 1, further comprising a strain relief collar having a first end to surround the fluid tubing of the constriction collar and an opposite end to surround at least some of said outer body.

12. The fluid port recited in claim 11, wherein said outer body has a taper that is surrounded by and held in locked engagement with the opposite end of said strain relief collar.

13. For connection to a patient implantable constriction collar to be positioned around a human organ and having an inflatable fluid chamber to which a fluid is added or from which a fluid is removed so as to generate a correspondingly increased or reduced occlusive pressure, and fluid tubing connected to the fluid chamber, the improvement of an implantable fluid port to be coupled to the fluid tubing of the constriction collar at which the fluid is supplied to or withdrawn from the fluid chamber thereof, said fluid port comprising:
an inner liner including a fluid reservoir to receive a needle cannula and a reservoir extension connected at one end thereof to said fluid reservoir and communicating at the opposite end thereof with the fluid tubing of the constriction collar, said reservoir extension being aligned at an angle with said fluid reservoir; and
an outer body surrounding said inner liner and including a suture attachment wing to receive a surgical suture for attaching said fluid port to the tissue of the patient and thereby preventing migration of said fluid port in relation to the constriction collar following implantation.

14. The fluid port recited in claim 13, wherein said outer body also includes a flat base spaced from said attachment wing, such that said flat base is positioned relative to said attachment wing so as to lie against one side of the patient's tissue to which said fluid port is attached following implantation, and said attachment wing is positioned to lie at the opposite side of the patient's tissue.

15. The fluid port recited in claim 13, further comprising a strain relief collar having a first end to surround the fluid tubing of the constriction collar and an opposite end to surround at least some of said outer body.

16. The fluid port recited in claim 13, wherein the fluid reservoir of said inner liner is aligned with said reservoir extension thereof at an angle of approximately 135 degrees.
